(19) United States
(12) Patent Application Publication
(10) Pub. No.: US 2008/0091228 A1
(43) Pub. Date: Apr. 17, 2008

(54) DOPPLER ENABLED SOFT TISSUE DISSECTOR

(75) Inventors: Gary Douglas, Billerica, MA (US); David Regan, Pelham, NH (US); Nilendu Srivastava, Chelmsford, MA (US); Rachana Suchdev, Nashua, NH (US); Ralph Clayman, Irvine, CA (US)

Correspondence Address: Vern Maine & Associates, 100 MAIN STREET, P O BOX 3445, NASHUA, NH, 03061-3445, US

(73) Assignee: VASCULAR TECHNOLOGY INC., Nashua, NH (US)

(21) Appl. No.: 11/869,264

(22) Filed: Oct. 9, 2007

Related U.S. Application Data
(60) Provisional application No. 60/828,480, filed on Oct. 6, 2006.

(51) Int. Cl. A61B 17/00

(52) U.S. Cl. 606/190

(57) ABSTRACT

A system for soft tissue dissection, the system comprising: an elongate handle, a Doppler crystal, a wire, partially disposed within the elongate handle, for transmitting signals to the Doppler crystal, a Doppler transmitter whereby the signals are generated, and a blunt tissue dissector tip.

### RELATED APPLICATIONS

This application claims the benefit of U.S. Provisional Applications No. 60/828,480, filed Oct. 6, 2006. This application is herein incorporated in its entirety by reference.

### FIELD OF THE INVENTION

The invention relates to soft tissue dissectors, and more particularly, to a soft tissue dissector configured for use in laparoscopy.

### BACKGROUND OF THE INVENTION

Soft tissue dissectors also known as Kittner dissectors, have been employed in open surgery at least since the Civil War. Such dissectors perform the double function of absorbing blood to improve visibility in the incision and to develop dissection planes between tissues. Other means used to induce such planes include digital or manual separation by the surgeon, a much cruder and often times less successful technique of blunt dissection.

Minimally invasive surgical techniques, such as laparoscopy and endoscopy have become increasingly prevalent as the benefits of decreased patient morbidity and recovery time have been recognized. These techniques, however, decrease access to the surgical site, requiring the surgeon to substitute direct digital contact with tissue, with thin-shafted laparoscopic probes or instruments that are over 4 inches in length. Likewise deteriorated, saturated, or rarely, lost dissectors must be replaced through small incisions, wasting time, increasing expense, and risking added morbidity. In addition, the Kittner as designed for open surgery assumes dissection in an often blood-tinged field; in contrast, with minimally invasive laparoscopic surgery, the pressure induced by the pneumoperitoneum, in addition to other clinical factors unique to minimally invasive surgery, largely preclude venous oozing and bleeding, thereby decreasing the need to clear the field of blood. Hence the absorbency of the Kittner, which results in saturation and deterioration of functionality, may work to the detriment of the laparoscopic surgeon.

Furthermore, the inability of a surgeon to digitally palpate the surgical field during minimally invasive procedures impedes the surgeon's ability to identify and evaluate vasculature, particularly subsurface vessels not readily visualized laparoscopically. Identification of the vasculature is particularly critical during dissection, in order to avoid inadvertent trauma to vessels. Instruments, such as laparoscopic Doppler probes, allow the laparoscopic surgeon to identify blood vessels and assess blood flow during minimally invasive procedures. Integration of Doppler function into a soft tissue dissector would allow a surgeon to dissect in vascular areas not only with increased knowledge of the underlying vasculature, but also with greater ease provided by a single, multi-functional instrument.

What is needed, therefore, are techniques for soft tissue dissection adapted for use in laparoscopic and endoscopic techniques. These dissectors would be light-weight, minimally absorbent, and durable, and would encompass Doppler functionality.

### SUMMARY OF THE INVENTION

One embodiment of the invention provides a system for soft tissue dissection, the system comprising: an elongated handle and a synthetic, minimally or non-absorbent, tip disposed on said handle.

Another embodiment of the present invention provides such a system wherein said tip is of minimally or non-absorbent foam.

Still another embodiment of the present invention provides such a system wherein said system further comprises a Doppler crystal; A wire, partially disposed within said elongate handle, for transmitting signals to said Doppler crystal; A Doppler transmitter whereby said signals are generated.

A still further embodiment of the present invention provides such a system wherein said tip is configured to expose said crystal.

Yet another embodiment of the present invention provides such a system wherein said tip is medical grade, minimally or non-absorbent, closed cell foam.

A yet further embodiment of the present invention provides such a system wherein said tip is from a material selected from the group of materials consisting of medical grade closed cell polyethylene, polyethylene/ethylene vinyl acetate co-polymers, combinations thereof, methylene diphenylene diisocyanate based flexible polyurethanes, and toluene diisocyanate based flexible polyurethanes.

A still further embodiment of the present invention provides such a system wherein said tip is of medical grade, non-absorbent vinyl.

Even another embodiment of the present invention provides such a system wherein said tip is sufficiently durable to resist deterioration throughout a surgical procedure.

An even further embodiment of the present invention provides such a system wherein said handle is configured from a material selected from metal, plastic, fiber glass, composites, and combinations thereof.

Another embodiment would equip the probe with an energy function for the dispersal of laser, ultrasonic, or electrosurgical energy for the purpose of coagulation of surface or deep arteries or veins in solid or hollow organs.

Another embodiment would equip the hollow probe with both suction and irrigation capabilities.

Another embodiment would provide for injection of sealing, biological glue, or hemostatic agents through the tip of the probe to control bleeding or hold two tissues together. In this case, the foam tip of the dissector could be made of a nonadherent material such that pressure could be applied to the bleeding surface of an organ immediately after the hemostatic agent or glue was administered. Other materials could likewise be instilled through the probe (e.g. analgesics, antiadhesion materials, antibiotic solution, etc.).

In another embodiment, the shape of the distal dissector could be altered by movement of an outer metal sheath or by internal changes due to deformation by an inner cell.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is an elevation view illustrating a soft tissue dissector configured in accordance with one embodiment of the present invention.

FIG. 2 is a table which details the major characteristics of three closed cell foams that can be used in the fabrication of a dissecting tip for a laparoscopic soft tissue dissector.

FIG. 3 is an elevation view illustrating a taper feature affixed to a soft tissue dissector in accordance with one embodiment of the present invention.

FIG. 4 is a perspective view illustrating a component that could be added to a soft tissue dissector to achieve a taper feature to a soft tissue dissector in accordance with one embodiment of the present invention.

FIG. 5 is an elevation view illustrating a Doppler enabled soft tissue dissector configured in accordance with one embodiment of the present invention.

FIG. 6A is a perspective view illustrating a cylindrical tip of a soft tissue dissector configured in accordance with one embodiment of the present invention.

FIG. 6B is a perspective view illustrating a frustoconical tip of a soft tissue dissector configured in accordance with one embodiment of the present invention.

FIG. 7 is a perspective view illustrating a Doppler enabled soft tissue dissector with a channel for the incorporation of an additional surgical modality or function.

FIG. 8 is a perspective view illustrating a spring loaded mechanism for the retraction of a Doppler crystal in accordance with one embodiment of the present invention.

FIG. 9 is a perspective view illustrating a Doppler enabled soft tissue dissector with an integrated injection needle.

### DETAILED DESCRIPTION

One embodiment of the present invention provides a soft tissue dissector 10 configured for use in minimally invasive surgical techniques such as laparoscopy and endoscopy. Such an embodiment utilizes a dissecting tip 12 disposed on a handle 14 configured for manipulation of the tip 12 through a surgical incision. The dissecting tip 12 is configured to be durable, and substantially non-absorbent.

It is desirable that, in such an embodiment, the dissecting tip 12 of the dissector be durable, securely mounted to the handle 14, and provide sufficient friction when applied to a bodily tissue without abrading the tissue. Such resistance allows the tip 12 to be used to dissect and move tissues and organs without slipping.

The handle 14 of the dissector 10 may be a straight rod, or may be configured with a curvature to permit the user greater range of motion. In an alternate embodiment, a curvature may be utilized to increase the ergonomics of surgical manipulation of the dissector. Likewise, one skilled in the art can appreciate that other handle configurations may be employed, for example handles that are configured to be flexed or bent by the user either before use or by external manipulation while inserted. The handle 14 may be configured from metal, plastic, fiber glass, composites, or other suitable material.

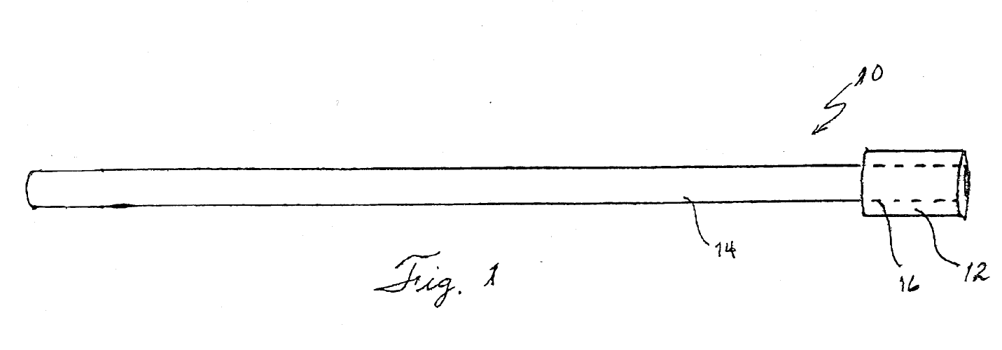

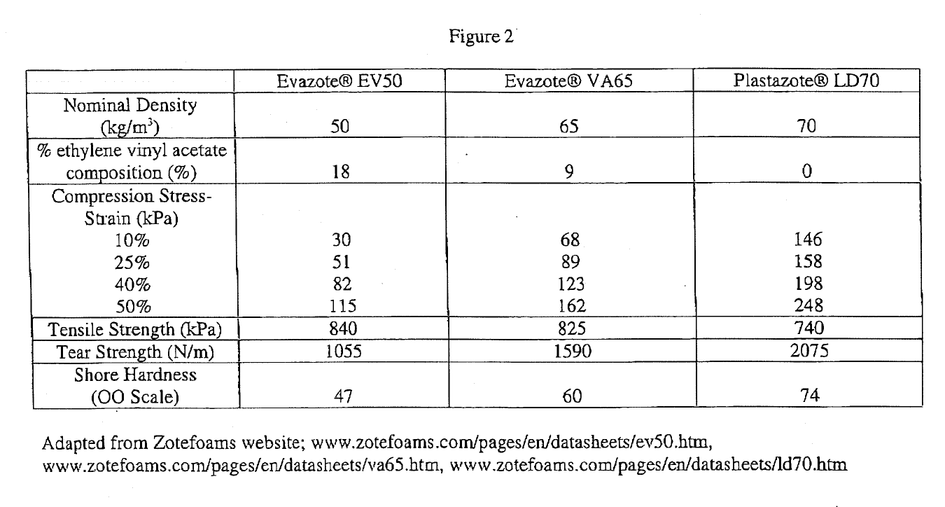

As illustrated in FIG. 1, a substantially non-absorbent soft tissue dissector 10 may be configured with an elongate handle 14, and a dissecting tip 12. The dissecting tip 12 may in one embodiment be affixed to the handle using a biocompatible epoxy 16. Likewise, other means of fixation, can be employed. In an alternate embodiment, the dissecting tip can be secured to a connector that attaches to a receiving connector secured to the handle, allowing for the interchange of tips during surgery. The material used in the dissecting tip may be a medical grade, non-absorbent, closed cell foam. Other materials such as medical grade vinyl, plastics or silicone may be employed. In one such embodiment medical grade closed cell polyethylene/ethylene vinyl acetate copolymer material is used. Similarly, polyethylene copolymers may be used without ethylene vinyl acetate. The foam is selected to have a rigidity and surface characteristics that allow it to resist excessive flexing without rupturing delicate tissues. In some embodiments, the foam would be configured with sufficient durability to survive a complete surgical procedure. Three particular closed cell foams, Evazote® EV50, Evazote® VA65 and Plastazote®LD70, have been identified and tested as ideal substrates for a substantially non-absorbent dissecting tip for laparoscopic use. FIG. 2 details the characteristics of these closed cell foams that are ideally suited in the fabrication of a substantially non-absorbent dissecting tip for a laparoscopic soft tissue dissector. In yet a further embodiment of the present invention, minimally absorbent open cell foams such as Novapreme™ (methylene diphenylene diisocyanate based flexible polyurethane) and Nolasponge™ (toluene diisocyanate based flexible polyurethane) could be utilized in the fabrication of a dissecting tip for a laparoscopic soft tissue dissector.

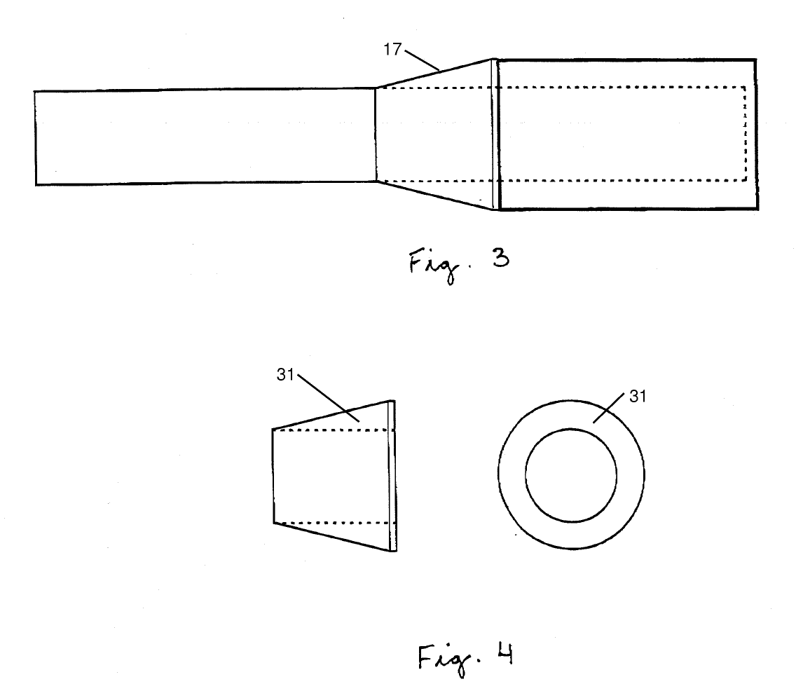

As illustrated in FIG. 3, in one embodiment of the present invention, a taper 17 from the dissecting tip 12 to the handle 14 is provided at the proximal end of the dissecting tip 12. This taper 17 functions to deflect mechanical forces that may be exerted on the dissecting tip 12 during removal of the soft tissue dissector 10 from a laparoscopic port or other similar surgical maneuvers. On occasion, the dissecting tip of currently used soft tissue dissectors detaches from the handle and falls into the surgical field, requiring retrieval. Removal of the soft tissue dissector from the laparoscopic port presents the greatest risk of detachment of the dissecting tip—if the dissector is not properly centered within the port channel during removal, the edge or wall of the port can exert excessive mechanical force on the dissecting tip. While retrieval of a relatively small component such as a dissecting tip may not be problematic in open surgical procedures, in laparoscopic procedures, the retrieval of such a component can be astonishingly time consuming and problematic. In fact, this complication can not only lead to increased procedure time and frustration, but also increased patient morbidity. The integration of a taper 17 just proximal to the dissecting tip 12 can provide a deflection mechanism against excessive mechanical force in these instances.

In addition, the taper 17 functions to prevent the insertion of the soft tissue dissector through an inappropriately small laparoscopic port. In one embodiment of the present invention, a dissecting tip 12 fabricated from foam would have a compressibility that could allow the insertion of the soft tissue dissector into an inappropriately small port channel. An appropriately sized non-compressible taper 17 at the proximal end of the dissecting tip 12 would prevent the insertion of the soft tissue dissector into an inappropriately small port channel. In one embodiment of the present invention, the taper 17 would have a diameter of 10 mm, for use of the soft tissue dissector through 10 mm laparoscopic ports. In other embodiments, the taper 20 would have a diameter of 12 mm, 5 mm or 2 mm for use through respectively sized laparoscopic ports.

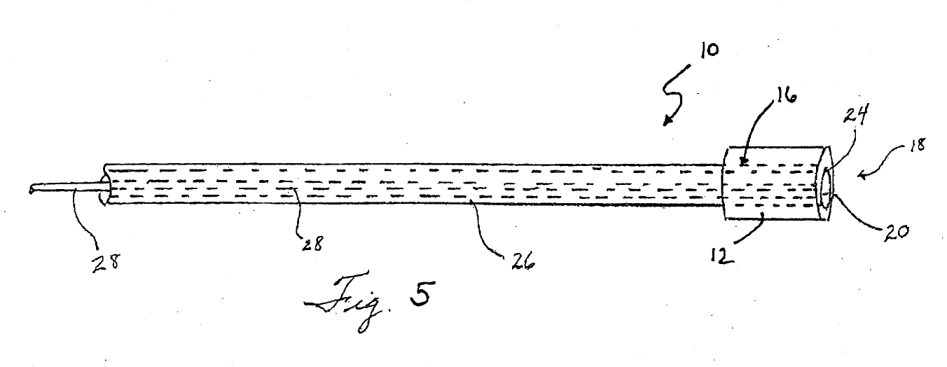

The taper 17 can be constructed from a biocompatible epoxy affixed to the handle 14 just proximal to the end of the dissecting tip 12. In another embodiment, the taper 17 can be affixed to both the handle 14 and the proximal end of the dissecting tip 12. In a still further embodiment, as illustrated in FIG. 4, the taper can be achieved using a distinct component 31 affixed to the handle just proximal to the dissecting tip (not shown). This component could be constructed from a rigid biocompatible material such as but not limited to stainless steel or plastic. Such a tapering component 25 would serve as a protective cap to the dissecting tip (not shown).

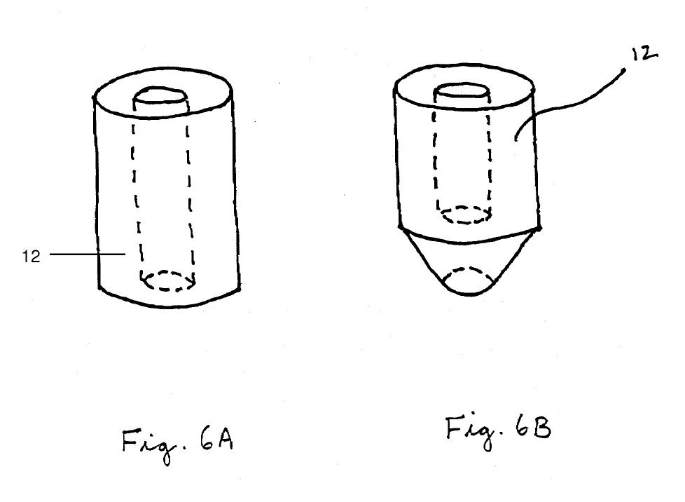

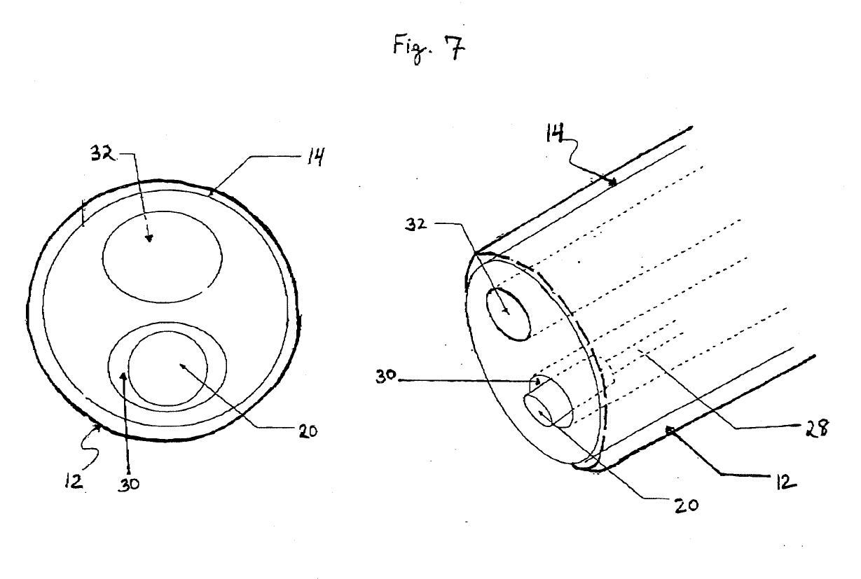

In an embodiment illustrated in FIG. 5, a dissector 10 is configured with a Doppler probe 18. The Doppler probe 18 comprises a piezo crystal 20 disposed within the tip of the dissector 10 such that it can be placed proximate to a tissue, for interrogation of that tissue with Doppler ultrasound waves. The probe may be utilized by the user to identify subsurface vessels not readily identifiable through a laparoscope. In one embodiment, the dissecting tip 12 would be configured with an aperture 24 in the distal end to permit the probe 18 to contact the tissue and the Doppler signal to be emitted without having to pass through the tip 12. Materials traditionally used in the fabrication of dissecting tips, such as tightly woven cotton, are not ideally suited for the incorporation of an aperture within the dissecting tip. As a result of the fabrication method used, these spherical tips are solid in nature; attempting to create an aperture through this material leads to a complete loss of spherical structure. Alternative materials that allow for the incorporation of an aperture must be identified. Such materials include but are not limited to synthetic foams and liquid vinyls. In one embodiment of the present invention, a medical grade closed cell foam such as, but not limited to, Evazote VA65 can be die-cut into a cylindrical shaped tip 12 as illustrated in FIG. 6A. Alternatively, a medical grade liquid vinyl, such as plastisol, can be dip molded to produce a frustoconical tip. A secondary operation utilizing a hole punch can be performed on the dip molded tip to provide an aperture through the distal end of the tip 12 as illustrated in FIG. 6B. In another embodiment, a medical grade foam with sufficient porosity, such as, but not limited to Nolasponge™, could be utilized in the fabrication of the dissecting tip, allowing the passage of Doppler signals through the foam material without an aperture, or a material could be selected with properties that would permit such signal transmission. A Doppler system suited for deployment in conjunction with the claimed invention is recited in co-pending U.S. application Ser. No. 11/014,037 which is hereby incorporated by reference for all purposes. Alternative embodiments utilize an 8 MHz Doppler without a plurality of preset depth settings.

In yet another embodiment of a laparoscopic Doppler enabled soft tissue dissector, a dissecting tip with sufficient durability to maintain its functionality through an entire surgical procedure would be desirable. Currently available soft tissue dissectors need to be replaced frequently because the absorbency of the material used in the fabrication of the dissecting tip leads to a loss of rigidity required for soft tissue dissection as the tip becomes saturated. Multiple kittners often need to be used through a single surgical procedure. In laparoscopic procedures, this leads to multiple exchanges of instruments through laparoscopic ports, increasing the length of such procedures. Furthermore, the incorporation of a Doppler within a soft tissue dissector would become cost prohibitive if multiple Doppler enabled dissectors needed to be used. Thus, a dissecting tip with greater durability is not only a desirable but also a critical requirement for the fabrication of a Doppler enabled soft tissue dissector. Identification of a substantially non-absorbent material for the fabrication of a durable dissecting tip is required. In one embodiment of the present invention, a durable closed cell foam could be used to achieve this requirement. Durability of a foam is dependent not only the material with which the foam is fabricated, but also the density of the particular material chosen. Foams with inadequate density will not maintain their structure with use. One closed cell foam that has been identified to have sufficient durability is Evazote® VA65. Other medical grade foams, vinyls, plastics or silicones may be employed. FIG. 2 details the characteristics of three foams that could provide sufficient durability in the fabrication of a dissecting tip for a laparoscopic Doppler enabled soft tissue dissector. In yet a further embodiment of the present invention, minimally absorbent open cell foams with sufficient durability such as Novapreme™ (methylene diphenylene diisocyanate based flexible polyurethane) and Nolasponge™ (toluene diisocyanate based flexible polyurethane) could be utilized in the fabrication of a dissecting tip for a laparoscopic Doppler enabled soft tissue dissector.

In one embodiment illustrated in FIG. 5, an elongate, fiber glass reinforced tube 26 is provided. A wire 28 for carrying signals to a Doppler crystal 20 is disposed within the tube. The Doppler crystal 20 is disposed at a distal end of the tube 26, the wire 28 passing through the tube 26 and exiting the proximal end of the tube 26, from whence it is connected to the Doppler transceiver (not shown). In such an embodiment, a switch (not shown) may be disposed at the proximal end of the tube 26 to allow the user to switch the Doppler signal on and off as the dissection process may cause distortions in the Doppler signals resulting in unreliable information. A protective tapering feature (not shown) can be affixed to the tube 26 just proximal to the dissecting tip 12. An epoxide layer 16 may be disposed between the dissecting tip 12 and the tube 26.

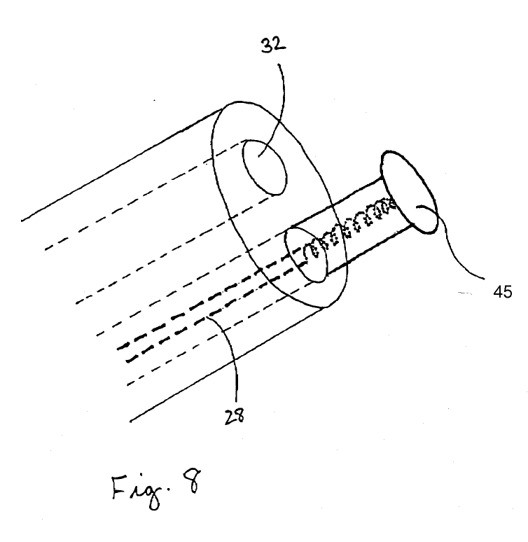

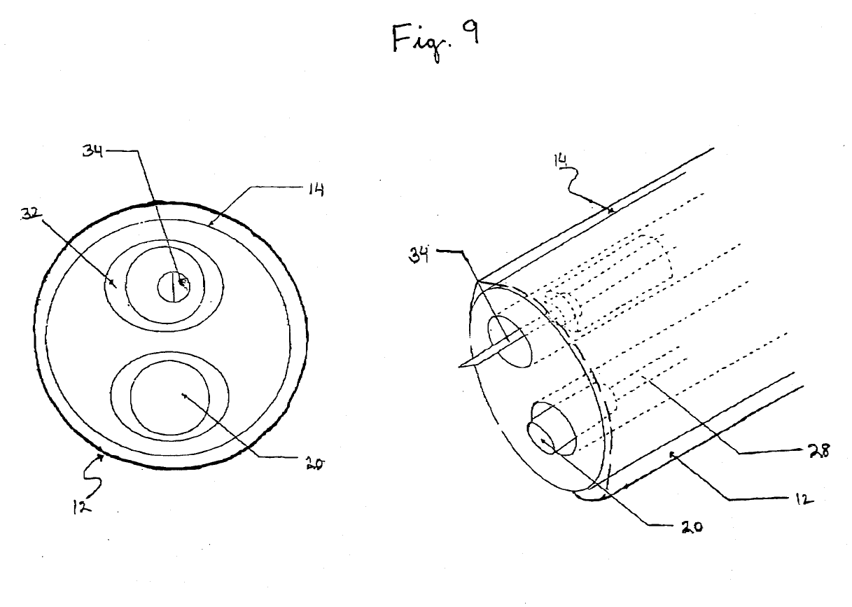

In an embodiment illustrated in FIG. 7, additional implements, modalities, or functions required during laparoscopic surgery can be integrated into a Doppler enabled dissector through the use of multiple channels disposed within the current elongate handle 14. One way to achieve multiple channels within the handle 14 is to extrude multilumen tubing using fiberglass composites such as but not limited to Lestran or rigid plastics such as HDPE. Alternatively, multi-lumen stainless steel tubing could be employed. While one channel 30 of the multilumen tubing could house the Doppler crystal 20 and connecting wire 28, additional channel(s) 32 could house additional implements, modalities or functions typically employed during laparoscopic surgery. In one such embodiment, the channel 32 within the handle may be used to introduce or aspirate fluids from the cavity, either those introduced into the cavity by the user or others generated by the body, to provide a Doppler enabled dissector with suction and irrigation capabilities. In such an embodiment, the channel(s) 32 is/are configured to carry sterile water, saline, and other liquids and is/are connected to a vacuum source. Alternatively such a channel 32 may also be configured to introduce and direct air or other gasses into the cavity. Such directed air flow may be used in dissection of tissues. Other functions can be integrated into channel(s) 32 within the handle, including mono or bipolar electrocautery units. Such units may comprise heating elements disposed within channel 32 at the distal end of the dissector, with power supplied via wires disposed within and exiting from the proximal end of the handle (not shown). In this embodiment, the Doppler crystal 20 would be configured to allow for retractability, such that the crystal could be withdrawn a safe distance into its channel 30 during heating or coagulation. Such an embodiment may also include a surgical grasper, configured in accord with known graspers, but disposed within a channel in the system configured according to the current invention. In one such embodiment, as illustrated in FIG. 8, a spring loaded advancer and retractor 45 connected to the Doppler wire 28 and operated by the user externally could be used to achieve this feature. Such integrated units may allow for the conduction of fluid, gas (e.g. argon) or energy to enhance dissection, coagulation, or other necessary actions during surgery.

In a still further embodiment of the invention and as shown in FIG. 9, the channel 32 could house an injection needle 34 that could be used for the injection of sealing agents or biological glue through the tip of the probe to control bleeding or hold two tissues together. In this case, the dissecting tip of the probe could be made of a nonadherent material such as silicone or coated foam such that pressure could be applied to the bleeding surface of an organ immediately after the sealing agent or glue was administered. Alternatively, hemostatic agents, such as epinephrine or sclerosing agents, could be injected to control bleeding. Other materials could likewise be instilled through the probe (e.g. analgesics, antiadhesion materials, antibiotic solution, etc.). In yet a further embodiment, the channel 32 could house a surgical grasper (not shown), to facilitate dissection of tissue.

In an alternative embodiment of the present invention, a modality for the delivery of therapeutic ultrasonic energy, such as high intensity focused ultrasound, could be housed in the channel 32 of a multilumen handle, with the Doppler mechanism housed in channel 30. This embodiment provides for a single device that can be used for dissection, detection of vessels and delivery of therapeutic energy. This energy may be used to coagulate Doppler identified blood vessels lying deep to the surface of an organ, to enhance enulceation of a tumor of said organ.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

Among that which is claimed is:

1. A system for delicate blunt tissue dissection, the system comprising:
An elongate handle;
A Doppler crystal;
A wire, partially disposed within said elongate handle, for transmitting signals to said Doppler crystal;
A Doppler transmitter whereby said signals are generated; and
A blunt tissue dissector tip.

2. The system of claim 1 wherein said blunt tissue dissector tip comprises non-absorbent foam.

3. The system of claim 1 wherein said blunt tissue dissector tip comprises medical grade vinyl.

4. The system of claim 1 wherein said tip is configured to expose said crystal.

5. The system of claim 1 further comprising at least one channel disposed within said elongate handle.

6. The system of claim 5 further comprising a vacuum source coupled to said channel.

7. The system of claim 5 further comprising a fluid source coupled to said channel, such that a fluid may be introduced through said channel into a surgical site.

8. The system according to claim 7 wherein said fluid is a fluid selected from the group of fluids consisting of liquids, gases, water, saline, air, pharmaceutical compounds, and solutions, suspensions and mixtures thereof.

9. The system of claim 1 wherein said handle is configured from a material selected from metal, plastic, fiber glass, composites, and combinations thereof.

10. The system according to claim 5 wherein said Doppler transmitter is retractable into said channel.

11. The system according to claim 5 further comprising a needle.

12. The system according to claim 11 wherein said needle is configured to introduce therapeutic agents into target tissues.

13. The system according to claim 5 further comprising a cautery disposed within said channel.

14. The system according to claim 5 further comprising a high intensity focused ultrasound transmitter disposed within said channel.

15. The system according to claim 5 further comprising a surgical grasper disposed within said channel.
